(19) United States
(12) Patent Application Publication
(10) Pub. No.: US 2026/0017761 A1
(43) Pub. Date: Jan. 15, 2026

(54) MAGNETIC RESONANCE IMAGE RECONSTRUCTION METHOD AND MAGNETIC RESONANCE IMAGE RECONSTRUCTING APPARATUS

(71) Applicant: CANON MEDICAL SYSTEMS CORPORAITON, Otawara-shi (JP)

(72) Inventors: Sha WANG, Beijing (CN); Lijun Zhang, Beijing (CN)

(73) Assignee: CANON MEDICAL SYSTEMS CORPORAITON, Otawara-shi (JP)

(21) Appl. No.: 19/269,136

(22) Filed: Jul. 15, 2025

(30) Foreign Application Priority Data
Jul. 15, 2024 (CN) 202410942500.0
Apr. 9, 2025 (JP) 2025-064220

(51) Int. Cl. G06T 5/60; A61B 5/055

(52) U.S. Cl. CPC G06T 5/60; A61B 5/055; G06T 2207/10088; G06T 2207/20084

(57) ABSTRACT

A magnetic resonance image reconstruction method according to an embodiment is a magnetic resonance image reconstruction method for reconstructing magnetic resonance image data on the basis of undersampled k-space data and includes: generating second image data by performing a regularization process within an image space while using a first neural network on first image data generated on the basis of the undersampled k-space data; generating third image data by correcting the second image data so that a pixel value statistical feature of the second image data approximates a pixel value statistical feature of the first image data; and generating fourth image data by performing a data integrity process on the third image data so that k-space data corresponding to the third image data approximates the undersampled k-space data.

### CROSS-REFERENCE TO RELATED APPLICATIONS

This application is based upon and claims the benefit of priority from Chinese Patent Application No. 202410942500.0, filed on Jul. 15, 2024; and Japanese Patent Application No. 2025-064220, filed on Apr. 9, 2025, the entire contents of all of which are incorporated herein by reference.

### FIELD

Embodiments described herein relate generally to a magnetic resonance image reconstruction method and a magnetic resonance image reconstructing apparatus.

### BACKGROUND

Magnetic resonance imaging technology is non-invasive medical image capturing technology that uses a magnetic resonance phenomenon where a hydrogen atom nucleus placed in a static magnetic field resonates with a radio frequency magnetic field having a specific frequency. The magnetic resonance imaging technology has advantages of having a high resolution, causing no trauma, and using no radiation. Because it is possible to perform an examination on a variety of parenchymal organs in the human body, the magnetic resonance imaging technology is widely applied to diagnosing clinical diseases.

To perform magnetic resonance imaging, k-space data for generating a magnetic resonance image is acquired by scanning an examined subject (a patient), so that the magnetic resonance image is reconstructed on the basis of the k-space data. However, acquiring all the k-space data requires scanning for a long period of time, which can easily cause discomfort for the patient and may increase the possibility of having motion artifacts. To cope with this problem, in magnetic resonance imaging, scanning time is usually shortened by acquiring partial k-space data through an undersampling process and further reconstructing a magnetic resonance image on the basis of undersampled k-space data.

When the magnetic resonance image is reconstructed on the basis of the undersampled k-space data, there is an insufficient amount of information for the reconstruction, and it is not possible to reconstruct ground truth for the magnetic resonance image. Thus, an estimation result of the ground truth for the magnetic resonance image is reconstructed. In other words, reconstructing the magnetic resonance image on the basis of the undersampled k-space data is an ill-posed problem, and it is not possible to reconstruct an accurate magnetic resonance image in one-to-one correspondence.

A magnetic resonance image reconstruction method is conventionally known by which a magnetic resonance image is reconstructed on the basis of undersampled k-space data by using a compressed sensing algorithm. According to this magnetic resonance image reconstruction method, an appropriate solution is determined in one-to-one correspondence, by adding a constraint to the reconstruction.

Following the development of machine learning technology in recent years, magnetic resonance image reconstruction methods using a neural network have been proposed. By having a regularization process performed within an image space or a k-space, it is possible to use the neural network for applying an appropriate constraint to reconstruction of a magnetic resonance image. As a magnetic resonance image reconstruction technique using a neural network, a method has been proposed by which a magnetic resonance image is reconstructed on the basis of undersampled k-space data by using an end-to-end unrolled reconstruction network. Advantages of this method include being able to reconstruct a magnetic resonance image with high quality and training thereof being convenient. In this situation, the reconstruction network is a large network in which a plurality of reconstruction modules each including a neural network are connected in series.

For instance, as a publicly-known example, a magnetic resonance image reconstruction method using an end-to-end unrolled reconstruction network has been proposed in which a reconstruction module included in the reconstruction network is provided with an image space regularization block for performing an image space regularization process by using a neural network and a data integrity block for performing a data integrity process. According to this publicly-known example, no processing is performed on input data or output data of the image space regularization block. Also, in another publicly-known example, a magnetic resonance image reconstruction method using an end-to-end unrolled reconstruction network has been proposed in which a reconstruction module included in a reconstruction network is provided with an image space regularization block and a data integrity block. In the latter publicly-known example, a normalization process is performed on input data to the image space regularization block, whereas an un-normalization process is performed on output data from the image space regularization block.

In the magnetic resonance image reconstruction using the end-to-end unrolled reconstruction network described above, when the neural networks included in the reconstruction network do not share parameters, because the neural networks have a higher degree of freedom for the parameters than when sharing the parameters, it is often the case that the reconstructed magnetic resonance image has a high level of precision. In contrast, when the neural networks included in a reconstruction network do not share parameters, there is a higher possibility that the neural networks may output unintended results. Thus, it is feared that the reconstructed magnetic resonance image may be completely different from ground truth, and a problem arises where the reconstruction of the magnetic resonance image has low stability.

In addition, the reconstruction networks in the publicly-known examples described above both have the problem where the image reconstruction has low stability, and there is a possibility that a magnetic resonance image that is completely different from ground truth may be reconstructed.

### BRIEF DESCRIPTION OF THE DRAWINGS

FIG. 1 is a diagram illustrating an exemplary configuration of a magnetic resonance image reconstructing apparatus according to a first embodiment;

FIG. 2 is a flowchart illustrating a flow in a magnetic resonance image reconstruction method according to the first embodiment;

FIG. 3 is a data flow chart for explaining a process at step S102 in the magnetic resonance image reconstruction method according to the first embodiment;

FIG. 4 is a data flow chart for explaining processes at steps S104 through S106 in the magnetic resonance image reconstruction method according to the first embodiment;

FIG. 5 is a diagram illustrating an exemplary configuration of a magnetic resonance image reconstructing apparatus according to a second embodiment;

FIG. 6 is a flowchart illustrating a flow in a magnetic resonance image reconstruction method according to the second embodiment;

FIG. 7 is a diagram illustrating an exemplary configuration of a magnetic resonance image reconstructing apparatus according to a comparison example;

FIG. 8 is a flowchart illustrating a flow in a magnetic resonance image reconstruction method according to the comparison example;

FIG. 9 is a drawing for explaining examples of regularized image data and revised image data generated by revision processes in the magnetic resonance image reconstruction method according to the comparison example;

FIG. 10 is a drawing for explaining examples of regularized image data and revised image data generated by revision processes in the magnetic resonance image reconstruction method according to the embodiment; and

FIG. 11 is a drawing for comparing a magnetic resonance image reconstructed by implementing the magnetic resonance image reconstruction method according to the embodiment, with a magnetic resonance image reconstructed by using the comparison example.

### DETAILED DESCRIPTION

A magnetic resonance image reconstruction method according to an embodiment is a magnetic resonance image reconstruction method for reconstructing magnetic resonance image data on the basis of undersampled k-space data and includes: generating second image data by performing a regularization process within an image space while using a first neural network on first image data generated on the basis of the undersampled k-space data; generating third image data by correcting the second image data so that a pixel value statistical feature of the second image data approximates a pixel value statistical feature of the first image data; generating fourth image data by performing a data integrity process on the third image data so that k-space data corresponding to the third image data approximates the undersampled k-space data; and outputting image data based on the fourth image data as the magnetic resonance image data that has been reconstructed.

Exemplary embodiments of a magnetic resonance image reconstruction method and a magnetic resonance image reconstructing apparatus will be explained below, with reference to the accompanying drawings.

### First Embodiment

A magnetic resonance image reconstructing apparatus according to a first embodiment is configured to perform an image reconstruction on image data in an image space, on the basis of undersampled k-space data acquired by scanning a patient. An object is to estimate ground truth image data corresponding to fully-sampled k-space data and to reconstruct magnetic resonance image data similar to the ground truth image data. Further, the k-space data is data obtained as a result of a magnetic resonance imaging apparatus transmitting a pulse signal to the patient positioned in a magnetic field that has been frequency encoded and phase encoded and further receiving an echo signal caused by a specific atomic nucleus magnetic resonance by using a plurality of receiver coils.

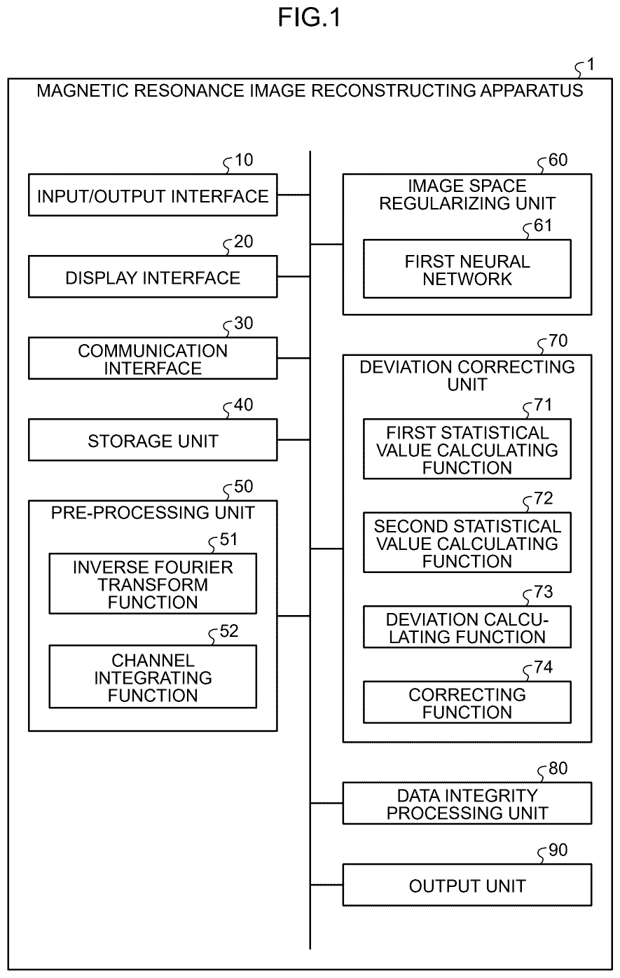

FIG. 1 is a chart illustrating an exemplary configuration of a magnetic resonance image reconstructing apparatus 1 according to the first embodiment. With reference to FIG. 1, the configuration of the magnetic resonance image reconstructing apparatus 1 according to the first embodiment will be explained. The magnetic resonance image reconstructing apparatus 1 according to the first embodiment includes an input/output interface 10, a display interface 20, a communication interface 30, a storage unit 40, a pre-processing unit 50, an image space regularizing unit 60, a deviation correcting unit 70, a data integrity processing unit 80, and an output unit 90. The input/output interface 10, the display interface 20, the communication interface 30, the storage unit 40, the pre-processing unit 50, the image space regularizing unit 60, the deviation correcting unit 70, the data integrity processing unit 80, and the output unit 90 are communicably connected to one another.

The input/output interface 10 is an interface for connecting the magnetic resonance image reconstructing apparatus 1 to an input apparatus (not illustrated) and is configured to receive an input operation of a user from the input apparatus and to transmit a signal based on the received input operation to the magnetic resonance image reconstructing apparatus 1. For example, the input/output interface 10 may be a serial bus interface such as a Universal Serial Bus (USB). Examples of the input apparatus include a mouse, a keyboard, a trackball, a switch, a button, a joystick, a touch screen, a microphone, and the like. Further, the input/output interface 10 may be connected to a storage apparatus so as to read and write various types of data to and from the storage apparatus. For example, the storage apparatus may be a Hard Disc Drive (HDD), a Solid State Drive (SSD), or the like.

The display interface 20 is an interface for connecting the magnetic resonance image reconstructing apparatus 1 to a display apparatus (not illustrated) and is configured to transmit data to the display apparatus so as to have an image displayed. For example, the display interface 20 may be a picture output interface such as Digital Visual Interface (DVI) or High-Definition Multimedia Interface (HDMI (registered trademark)). Examples of the display apparatus include a Liquid Crystal Display (LCD) or an organic Electroluminescence (EL) display. The display apparatus is configured to display a user interface for receiving an input operation from the user and the magnetic resonance image data output from the magnetic resonance image reconstructing apparatus 1, or the like. For example, the user interface may be a Graphical User Interface (GUI).

The communication interface 30 is an interface for connecting the magnetic resonance image reconstructing apparatus 1 to a server (not illustrated) and is capable of transmitting and receiving various types of data to and from the server. For example, the communication interface 30 may be a network card such as a wireless network card or a wired network card.

The storage unit 40 is configured to store therein image data used for the image reconstruction and user data such as the k-space data. Further, the storage unit 40 is configured to store therein a parameter used when the magnetic resonance image reconstructing apparatus 1 performs the image reconstruction such as a parameter for a neural network, for example. Further, the storage unit 40 is configured to store therein training data for training neural networks and other learnable parameters used by the magnetic resonance image reconstructing apparatus 1. For example, the storage unit 40 may be realized by using a storage apparatus such as a Read-Only Memory (ROM), a flash memory, a Random Access Memory (RAM), a Hard Disc Drive (HDD), a Solid State Drive (SSD), or a register. The flash memory, the HDD, and the SSD among others are each a non-volatile storage medium. These non-volatile storage media may each be realized by using another storage apparatus connected via a network, such as a Network Attached Storage (NAS) or an external storage server apparatus. In this situation, examples of the abovementioned network include the Internet, a Wide Area Network (WAN), a Local Area Network (LAN), a carrier terminal, a wireless communication network, a wireless base station, a dedicated line, and the like.

The pre-processing unit 50 is configured to generate initial image data by pre-processing the undersampled k-space data, which is input data of the magnetic resonance image reconstructing apparatus 1. The pre-processing unit 50 includes an inverse Fourier transform function 51 and a channel integrating function 52. The inverse Fourier transform function 51 is configured to perform an inverse Fourier transform on data by using an algorithm of an inverse fast Fourier transform or the like. The channel integrating function 52 is configured to integrate data of multiple channels respectively corresponding to the receiver coils of the magnetic resonance imaging apparatus, into data of a single channel.

By performing a regularization process within the image space while using a first neural network 61 on input image data, the image space regularizing unit 60 is configured to generate regularized image data, which is image data resulting from the regularization process. The image space regularizing unit 60 includes the first neural network 61. For example, the first neural network 61 may be a feed forward neural network, a convolutional neural network, or a transformer. The first neural network 61 may preferably be a convolutional neural network. Even more preferably, the first neural network 61 may be a U-Net. In the present embodiment, it is assumed that the first neural network 61 is a convolutional neural network including an input layer, an output layer, a convolution layer, an activation layer, a pooling layer, a batch normalization layer, and a fully-connected layer, while the size of the input layer is equal to that of the output layer. The first neural network 61 is configured to realize a function to perform an image space regularization process, by having loaded therein a neural network parameter dedicated for the first neural network 61 and stored in the storage unit 40. By loading various neural network parameters therein, it is possible to cause the first neural network 61 to perform various regularization processes.

The deviation correcting unit 70 is configured to generate deviation corrected image data resulting from a deviation correction, by correcting the regularized image data so that a pixel value statistical feature of the regularized image data generated by the image space regularizing unit 60 approximates a pixel value statistical feature of the initial image data corresponding to the undersampled k-space data. The deviation correcting unit 70 includes a first statistical value calculating function 71, a second statistical value calculating function 72, a deviation calculating function 73, and a correcting function 74. The first statistical value calculating function 71 is configured to calculate a first statistical value related to the initial image data on the basis of pixel values of the initial image data. The second statistical value calculating function 72 is configured to calculate a second statistical value related to the regularized image data, on the basis of pixel values of the regularized image data. The deviation calculating function 73 is configured to calculate a deviation value between the initial image data and the regularized image data, on the basis of the first statistical value and the second statistical value. The correcting function 74 is configured to generate the deviation corrected image data by correcting the regularized image data while using the calculated deviation value.

The data integrity processing unit 80 is configured to generate revised image data by performing a data integrity process on the deviation corrected image data so that k-space data corresponding to the deviation corrected image data approximates the undersampled k-space data.

The output unit 90 is configured to output image data based on the revised image data, as reconstructed magnetic resonance image data.

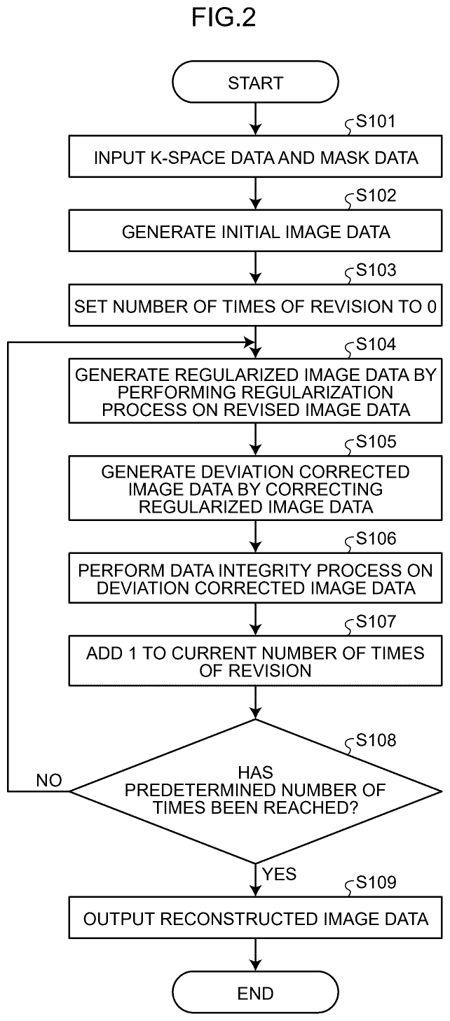

FIG. 2 is a flowchart illustrating a flow in a magnetic resonance image reconstruction method according to the first embodiment. The following will describe the flow in the magnetic resonance image reconstruction method according to the first embodiment, with reference to FIG. 2.

To begin with, the process proceeds to step S101.

At step S101, according to the user interface displayed on the display apparatus, the user selects, via the input apparatus, undersampled k-space data K₀ and mask data M that either are stored in the storage unit 40 or have been input from an external storage apparatus and further inputs the selected data to the magnetic resonance image reconstructing apparatus 1.

The following explanation will be based on that the k-space data K₀ is three-dimensional tensor data corresponding to a width W×a height H×the number of channels (the number of receiver coils) C, while the width direction corresponds to a frequency encoding direction, and the height direction corresponds to a phase encoding direction. In a magnetic resonance scan, an undersampling process is performed by omitting specific frequency encoding and specific phase encoding so as to reduce scanning time. For this reason, during the magnetic resonance scan, the scan is performed by skipping certain coordinates corresponding to the specific frequency encoding and the specific phase encoding. As a result, in the k-space data K₀, there is no data for a certain part of the coordinates. Thus, zero padding is performed for the data at the certain part of the coordinates. Because the data in the vicinity of the center position of k-space data has a large impact on contrast of reconstructed image data, it is a common practice during the undersampling process to intensively sample data from the vicinity of the center position in terms of the frequency encoding direction and the phase encoding direction, while skipping the data in some of the positions distant from the center position.

The mask data M indicates, with respect to the magnetic resonance scan, which coordinates were sampled and which coordinates were omitted in the k-space data K₀. For instance, let us discuss an example in which the mask data M is a matrix having the width W ×the height H, while the width direction corresponds to the frequency encoding direction, and the height direction corresponds to the phase encoding direction. In the mask data M, the values at the coordinates where the frequency encodes and the phase encodes that were sampled are positioned are each 1, whereas the values at the coordinates where the frequency encodes and the phase encodes that were not sampled are positioned are each 0.

When the process at step S101 is completed, the process proceeds to step S102.

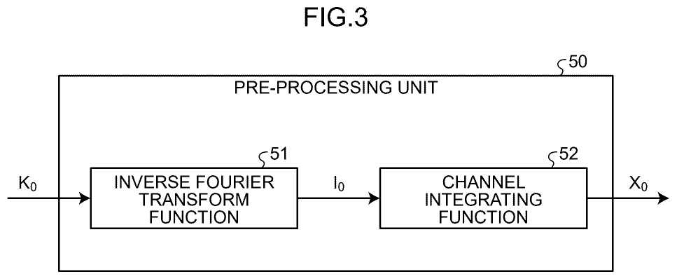

At step S102, the pre-processing unit 50 generates initial image data X₀ on the basis of the undersampled k-space data K₀. The following will explain a process at step S102 with reference to FIG. 3. FIG. 3 is a data flow chart for explaining the process at step S102 in the magnetic resonance image reconstruction method according to the first embodiment. In FIG. 3, the flow of the data is indicated with the solid arrows.

To begin with, the pre-processing unit 50 reads the k-space data K₀ from the storage unit 40 and, by employing the inverse Fourier transform function 51, performs an inverse Fourier transform on the k-space data K₀ so as to generate multi-channel image space data I₀. The multi-channel image space data I₀ is image space data of which the width, the height, and the number of channels are the same as those of the K-space data K₀. In the multi-channel image space data I₀, each of the pieces of data corresponding to the channels is image space data transformed from the k-space data acquired by a corresponding one of the receiver coils.

Subsequently, by employing the channel integrating function 52, the pre-processing unit 50 generates the initial image data X₀ by integrating, into data corresponding to a single channel, the data of the plurality of channels in the multi-channel image space data I₀, in accordance with sensitivity of each of the receiver coils. In this situation, the initial image data X₀ is two-dimensional image data corresponding to the width W×the height H that was directly generated on the basis of the undersampled k-space data K₀ and therefore presents problems such as having a lot of artifacts and noise, while details are missing and the image is blurry.

When the process at step S102 is completed, the process proceeds to step S103.

At steps S103 through S108, a revision process is repeatedly performed on the image data as many times as a predetermined number, on the basis of the initial image data X₀. In this situation, the predetermined number of times may preferably be eight to ten times. The image data resulting from the revision process performed t times (where t is an integer of 0 or larger) on the basis of the initial image data X₀ will hereinafter be referred to as revised image data Xₜ. When t=0, the revised image data X₀ represents the same image as that represented by the initial image data X₀.

At step S103, the magnetic resonance image reconstructing apparatus 1 sets the current number of times of revision (the number of times of iteration) to 0. When the process at step S103 is completed, the process proceed to step S104.

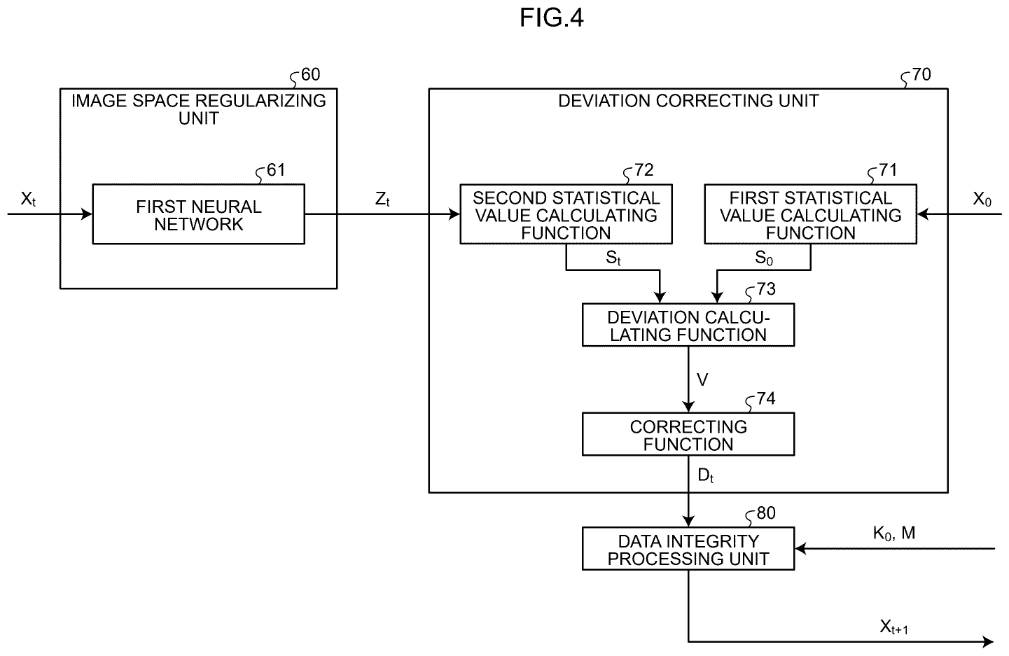

Next, processes at steps S104 through S106 will be explained, with reference to FIG. 4. FIG. 4 is a data flow chart for explaining the processes at steps S104 through S106 in the magnetic resonance image reconstruction method according to the first embodiment. In FIG. 4, the flow of the data is indicated with the solid arrows.

At step S104, the image space regularizing unit 60 generates regularized image data Zₜ, by performing a regularization process within the image space while using the first neural network 61 on the revised image data Xₜ. In this situation, t denotes the current number of times of revision.

To begin with, the image space regularizing unit 60 reads, from the storage unit 40, a neural network parameter for the first neural network 61 corresponding to the current number of times of revision and loads the parameter into the first neural network 61. Further, the image space regularizing unit 60 reads the revised image data Xₜ from the storage unit 40 and inputs the revised image data Xₜ to the first neural network 61 into which the parameter has already been loaded. After that, the image space regularizing unit 60 causes the first neural network 61 to execute forward propagation based on the revised image data Xₜ and thus calculates regularized image data Zₜ, which is output data of the first neural network 61. In this situation, the regularized image data Zₜ is two-dimensional image data having the same size as the revised image data Xₜ.

The processes performed by the first neural network 61 may be considered as a noise removing process, an artifact removing process, and an anti-alias process performed on the revised image data Xₜ. Accordingly, the regularized image data Zₜ may be considered as a result of performing the noise removing process, the artifact removing process, and the anti-alias process on the revised image data Xₜ. It is desirable to configure the first neural network 61 to use mutually-different neural network parameters for revision processes corresponding to mutually-different number of times of revisions. By applying the mutually-different parameters to the first neural network 61 at mutually-different revision stages, it is possible to perform appropriate processes on the revision image data Xₜ corresponding to the mutually-different revision stages.

When the process at step S104 is completed, the process proceeds to step S105.

At step S105, the deviation correcting unit 70 generates deviation corrected image data Dₜ, by correcting the regularized image data Zₜ, so that a pixel value statistical feature of the regularized image data Zₜ generated at step S104 approximates a pixel value statistical feature of the initial image data X₀.

To begin with, the deviation correcting unit 70 calculates a first statistical value S₀ related to the initial image data X₀ on the basis of pixel values of the initial image data X₀ by employing the first statistical value calculating function 71, and calculates a second statistical value Sₜ related to the regularized image data Zₜ on the basis of pixel values of the regularized image data Zₜ by employing the second statistical value calculating function 72. In this situation, the first statistical value So and the second statistical value Sₜ are statistical values of mutually the same type. For example, as the first statistical value S₀ and the second statistical value Sₜ, it is possible to use any of the following of the pixel values in the image data: an average value, a standard deviation, a median, a minimum value, a maximum value, and the like.

Subsequently, by employing the deviation calculating function 73, the deviation correcting unit 70 calculates a value obtained by dividing the second statistical value Sₜ by the first statistical value S₀, as a deviation value V.

Lastly, by employing the correcting function 74, the deviation correcting unit 70 calculates, as the deviation corrected image data Dₜ, a value obtained by dividing the regularized image data Zₜ by the deviation value V. In this situation, the deviation corrected image data Dₜ has a pixel value statistical feature closer to the initial image data X₀, as compared to the regularized image data Zₜ.

When the process at step S105 is completed, the process proceeds to step S106.

At step S106, the data integrity processing unit 80 generates revised image data Xₜ₊₁ by performing a data integrity process on the deviation corrected image data Dₜ so that the k-space data corresponding to the deviation corrected image data Dₜ approximates the undersampled k-space data K₀.

The data integrity processing unit 80 calculates the revised image data Xₜ₊₁ by using Expression (1) presented below:

$${X}_{t + 1} = arg min λ {‖ A ( {X}_{t + 1} ) - {K}_{0} ‖}_{2}^{2} + {‖ {X}_{t + 1} - {D}_{t} ‖}^{2} \qquad ( 1 )$$

In Expression (1), λ is a data integrity coefficient, whereas S is a forward operator. The value λ may be a fixed value being set in advance or may be a value that can be trained. An operation A(X) performed on arbitrary image data X by using the forward operator A denotes acquiring k-space data corresponding to the image data X and performing a Fourier transform thereon with the transform of the image data X into image data of multiple channels, and further calculating the Hadamard product of the acquired k-space data and the mask data M.

It is possible to solve Expression (1) by using an optimization algorithm based on a gradient descent scheme, a proximal mapping scheme, or the like. In this situation, the proximal mapping scheme may be solved by further using a conjugate gradient scheme.

In the process described above, pixel values of the revised image data Xₜ₊₁ generated from the data integrity process are close to pixel values of the deviation corrected image data Dₜ. In contrast, the k-space data corresponding to the revised image data Xₜ₊₁ is closer to the undersampled k-space data K₀, as compared to the k-space data corresponding to the deviation corrected image data Dₜ.

When the process at step S106 is completed, the process proceeds to step S107.

At step S107, 1 is added to the current number of times of revision.

In the magnetic resonance image reconstruction method according to the present embodiment, the revised image data Xₜ₊₁ is generated by performing the revision process once on the revised image data Xₜ through the processes at steps S104 to S107. In this situation, the revised image data Xₜ₊₁ is image data closer to ground truth image data, as compared to the revised image data Xₜ.

When the process at step S107 is completed, the process proceeds to step S108.

At step S108, it is judged whether or not the current number of times of revision has reached the predetermined number of times. When it is determined that the predetermined number of times has been reached, the process proceeds to step S109. On the contrary, when it is determined that the predetermined number of times has not been reached, the process proceeds to step S104.

At step S109, the revised image data resulting from the revision performed the predetermined number of times is output as estimated values with respect to the ground truth image data.

When the process at step S109 is completed, the processes in the magnetic resonance image reconstruction method are finished.

In this situation, the processes at steps S104 through S106 may be considered as a revision process performed once on the revised image data Xₜ while using a revision module including revision processes performed by an image space regularizing block, a deviation correcting block, and a data integrity block. Among these processes, the processes at steps S104 through S106 correspond to the processes performed by the image space regularizing block, the deviation correcting block, and the data integrity block, respectively.

Further, the processes at steps S103 through S109 may be considered as a process of generating the estimated values with respect to the ground truth image data, by processing the initial image data X₀, while employing a reconstruction network including a plurality of revision modules connected in series, i.e., while employing an end-to-end unrolled reconstruction network.

### Second Embodiment

Next, a magnetic resonance image reconstruction method and a magnetic resonance image reconstructing apparatus according to a second embodiment will be explained. In the second embodiment, differences from the first embodiment will primarily be explained, while explanations of some of the elements shared with the first embodiment will be omitted. In the description of the second embodiment, some of the elements that are the same as those in the first embodiment will be referred to by using the same reference characters.

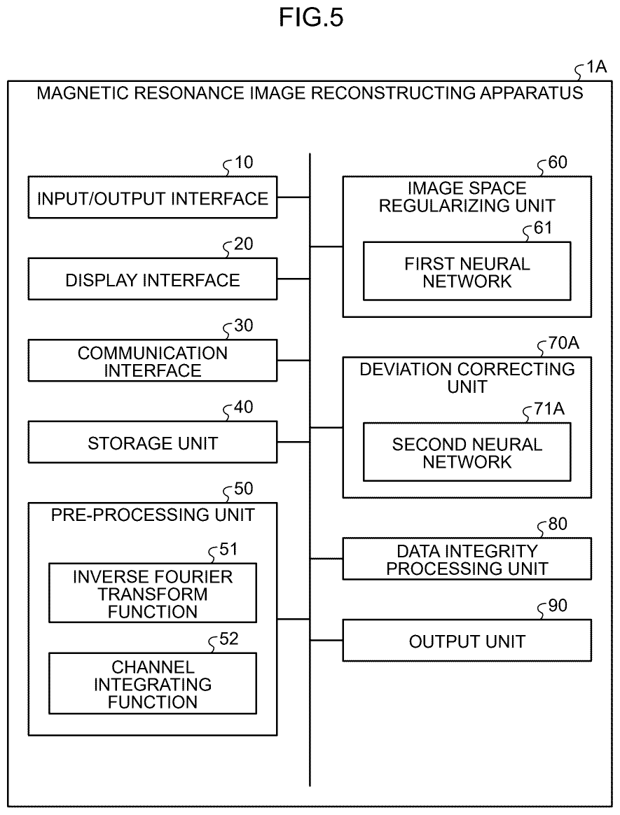

FIG. 5 is a diagram illustrating an exemplary configuration of a magnetic resonance image reconstructing apparatus 1A according to the second embodiment. The configuration of the magnetic resonance image reconstructing apparatus 1A according to the second embodiment will be explained, with reference to FIG. 5. In comparison to the magnetic resonance image reconstructing apparatus 1 according to the first embodiment, the magnetic resonance image reconstructing apparatus 1A according to the second embodiment includes a deviation correcting unit 70A in place of the deviation correcting unit 70.

The deviation correcting unit 70A is configured to generate deviation corrected image data resulting from a deviation correction, by correcting the regularized image data so that a pixel value statistical feature of the regularized image data generated by the image space regularizing unit 60 approximates a pixel value statistical feature of the initial image data corresponding to the undersampled k-space data. The deviation correcting unit 70A includes a second neural network 71A. For example, the second neural network 71A may be a feed forward neural network, a convolutional neural network, a transformer, or the like. The second neural network 71A may preferably be a convolutional network. Even more preferably, the second neural network 71A may be a U-Net. In the present embodiment, it is assumed that the second neural network 71A is a convolutional neural network including an input layer, an output layer, a convolution layer, an activation layer, a pooling layer, a batch normalization layer, and a fully-connected layer, while the size of the input layer is equal to that of the output layer. The second neural network 71A is configured to realize a function to perform a deviation correcting process, by having loaded therein a neural network parameter dedicated for the second neural network 71A and stored in the storage unit 40.

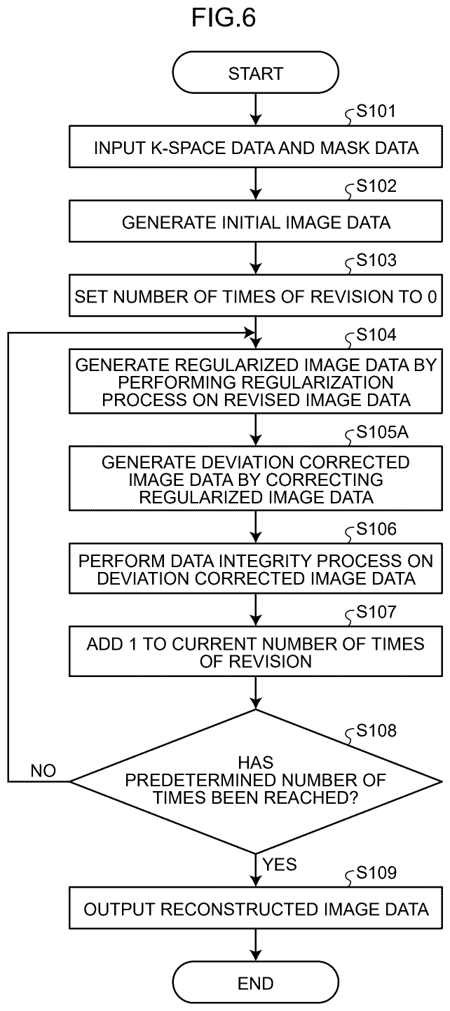

FIG. 6 is a flowchart illustrating a flow in a magnetic resonance image reconstruction method according to the second embodiment. The flow in the magnetic resonance image reconstruction method according to the second embodiment will be explained, with reference to FIG. 6. In comparison to the magnetic resonance image reconstruction method according to the first embodiment, the magnetic resonance image reconstruction method according to the second embodiment includes a step S105A in place of step S105.

In the second embodiment, when the process at step S104 is completed, the process proceeds to step S105A.

At step S105A, the deviation correcting unit 70A generates deviation corrected image data Dₜ, by correcting the regularized image data Zₜ, so that a pixel value statistical feature of the regularized image data Zₜ generated at step S104 approximates a pixel value statistical feature of the initial image data X₀.

To begin with, the deviation correcting unit 70A reads the neural network parameter for the second neural network 71A from the storage unit 40 and causes the second neural network 71A to load the parameter therein. After that, together with the regularized image data Zₜ, the deviation correcting unit 70A inputs the initial image data X₀ to the second neural network 71A into which the parameter has already been loaded. Further, the deviation correcting unit 70A causes the second neural network 71A to execute forward propagation based on the initial image data X₀ and the regularized image data Zₜ and thus calculates the deviation corrected image data Dₜ, which is output data of the second neural network 71A.

When the process at step S105A is completed, the process proceeds to step S106.

### Comparison Example

Next, a magnetic resonance image reconstruction method and a magnetic resonance image reconstructing apparatus according to a comparison example using a conventional technique will be explained. As for the comparison example, differences from the first embodiment will primarily be explained, while explanations of some of the elements shared with the first embodiment will be omitted. In the description of the comparison example, some of the elements that are the same as those in the first embodiment will be referred to by using the same reference characters.

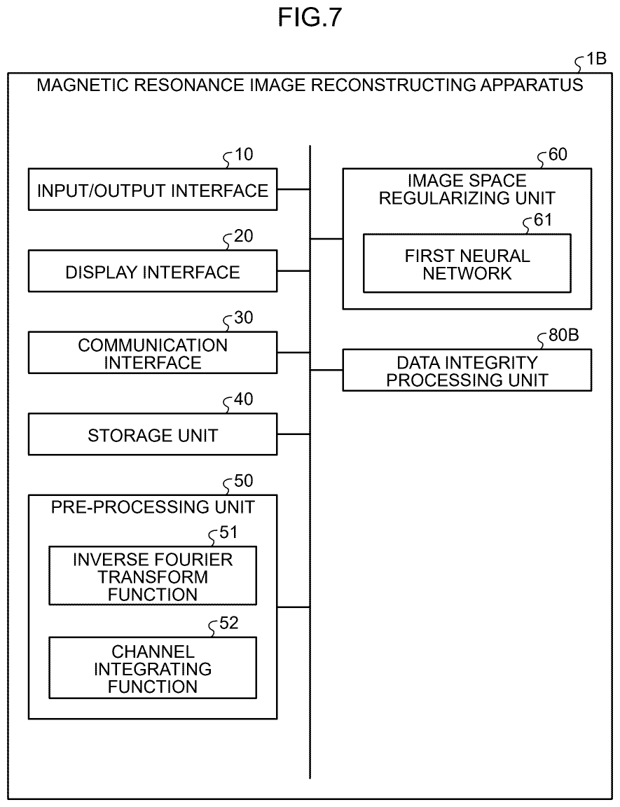

FIG. 7 is a diagram illustrating an exemplary configuration of a magnetic resonance image reconstructing apparatus 1B according to the comparison example. The configuration of the magnetic resonance image reconstructing apparatus 1B according to the comparison example will be explained, with reference to FIG. 7. In comparison to the magnetic resonance image reconstructing apparatus 1 according to the first embodiment, the magnetic resonance image reconstructing apparatus 1B according to the comparison example does not include the deviation correcting unit 70 and includes a data integrity processing unit 80B in place of the data integrity processing unit 80.

The data integrity processing unit 80B is configured to generate revised image data by performing a data integrity process on the regularized image data so that the k-space data corresponding to the regularized image data approximates the undersampled k-space data.

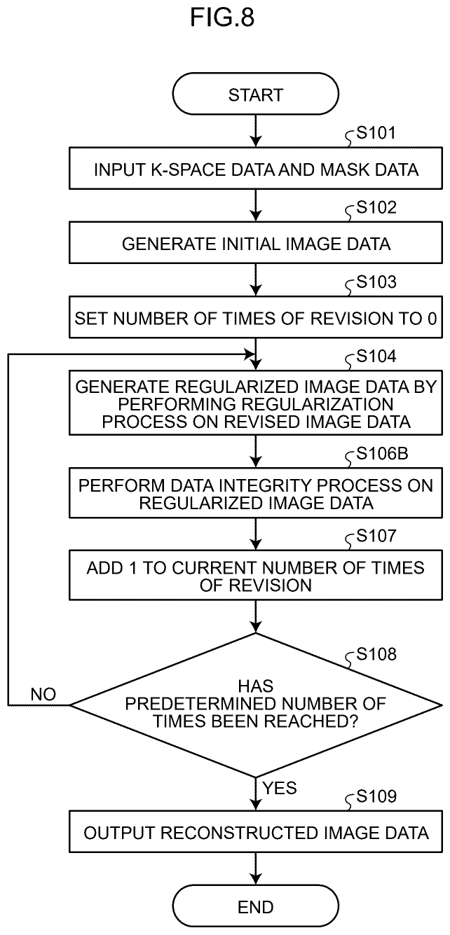

FIG. 8 is a flowchart illustrating a flow in the magnetic resonance image reconstruction method according to the comparison example. The flow in the magnetic resonance image reconstruction method according to the comparison example will be explained, with reference to FIG. 8. In comparison to the magnetic resonance image reconstruction method according to the first embodiment, the magnetic resonance image reconstruction method according to the comparison example does not include step S105 and includes step S106B in place of step S106.

In the comparison example, when the process at step S104 is completed, the process proceeds to step S106B.

At step S106B, the data integrity processing unit 80B generates revised image data Xₜ₊₁ by performing a data integrity process on the regularized image data Zₜ so that the k-space data corresponding to the regularized image data Zₜ approximates the undersampled k-space data K₀.

The data integrity processing unit 80B calculates the revised image data Xₜ₊₁ by using Expression (2) presented below.

$${X}_{t + 1} = arg min λ {‖ A ( {X}_{t + 1} ) - {K}_{0} ‖}_{2}^{2} + {‖ {X}_{t + 1} - {Z}_{t} ‖}^{2} \qquad ( 2 )$$

In Expression (2), λ is a data integrity coefficient, whereas A is a forward operator. It is possible to solve Expression (2) by using an optimization algorithm based on a gradient descent scheme, a proximal mapping scheme, or the like. In this situation, the proximal mapping scheme may be solved by further using a conjugate gradient scheme.

When the process at step S106B is completed, the process proceeds to step S107.

### Technical Advantageous Effects

Next, advantageous effects of the magnetic resonance image reconstruction method and the magnetic resonance image reconstructing apparatus according to the embodiment will be explained.

According to the magnetic resonance image reconstruction technique based on the end-to-end unrolled reconstruction network, the estimated vales with respect to the ground truth image data are generated by performing the revision multiple times on the initial image data. In this situation, when the regularization process is performed on the image data by employing a neural network, the neural network might be unable to correctly process the image data and might add artifacts or noise to the image or might make the image blurry in the regularization process. Thus, there would be a possibility that the regularized image data generated by the regularization process might be different from an intrinsic distribution.

In the comparison example, after the regularized image data is generated by using the neural network, the revised image data is generated by performing the data integrity process while using the regularized image data itself as is, without processing the regularized image data. For this reason, if the regularized image data generated in a revision process at any of the stages data is different from the intrinsic distribution, the revised image data generated on the basis of the regularized image data will also be different from an intrinsic distribution. In addition, because the neural network is unable to correctly process the image data different from the intrinsic distribution, the revision processes thereafter will be unable to correctly revise the image data. In this manner, artifacts and noise become more and more serious as the revision processes continue, and the precision level of the reconstructed magnetic resonance image will be greatly degraded.

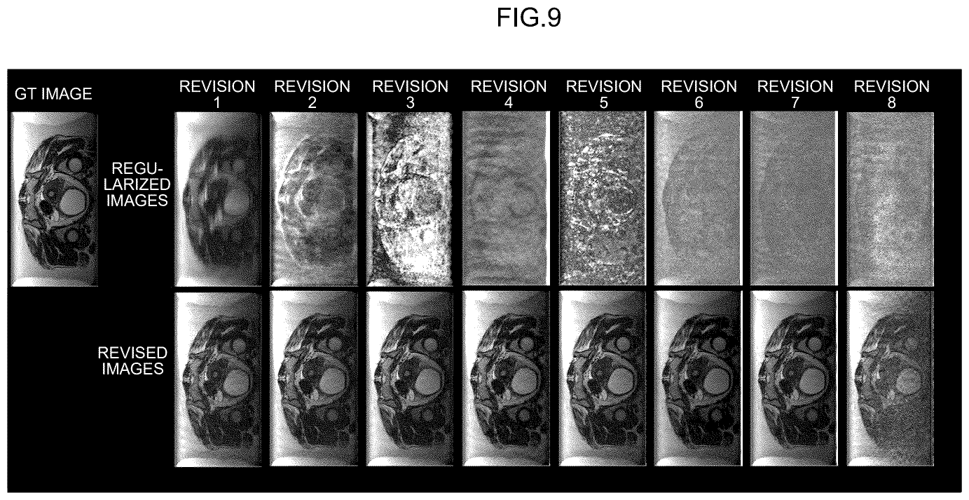

FIG. 9 is a drawing for explaining examples of the regularized image data and the revised image data generated by the revision processes in the magnetic resonance image reconstruction method according to the comparison example. Problems of the magnetic resonance image reconstruction method according to the comparison example will be explained, with reference to FIG. 9.

FIG. 9 illustrates an example in which a magnetic resonance image related to an axial plane in the vicinity of the prostate is reconstructed by implementing the magnetic resonance image reconstruction method according to the comparison example. As illustrated in FIG. 9, in the magnetic resonance image reconstruction method according to the comparison example, the revision process is performed eight times in total. Among those, the regularized image data generated in the revision process performed for the second time exhibits artifacts and noise caused by the neural network. Subsequently, in the revision processes performed for the third to the eighth times, the neural network becomes unable to accurately perform the regularization process due to impacts of the artifacts and the noise, and the generated regularized image data increasingly becomes different from the ground truth image data. Lastly, in the revision process performed for the eighth time, because the revised image data is generated on the basis of the regularized image data greatly different from the ground truth image data, the generated revised image data similarly exhibits artifacts and noise, and the precision level of the generated revised image data is greatly degraded.

In contrast, in the embodiment, after the regularized image data is generated by employing the neural network, the deviation corrected image is generated by performing the deviation correction on the regularized image data on the basis of the pixel value statistical feature of the initial image data and further generates the revised image data by performing the data integrity process while using the deviation corrected image. As a result, even when regularized image data different from the intrinsic distribution is generated in a revision process at one of the stages, it is possible to prevent the revised image data from becoming different from the intrinsic distribution. According to the embodiment, it is possible to prevent the precision level of the reconstructed magnetic resonance image from being greatly degraded and to enhance stability of the magnetic resonance image reconstruction. Furthermore, in the embodiment, because it is also possible to increase convergence speed of the revised image data, it is possible to reduce the number of times the revision process needs to be performed.

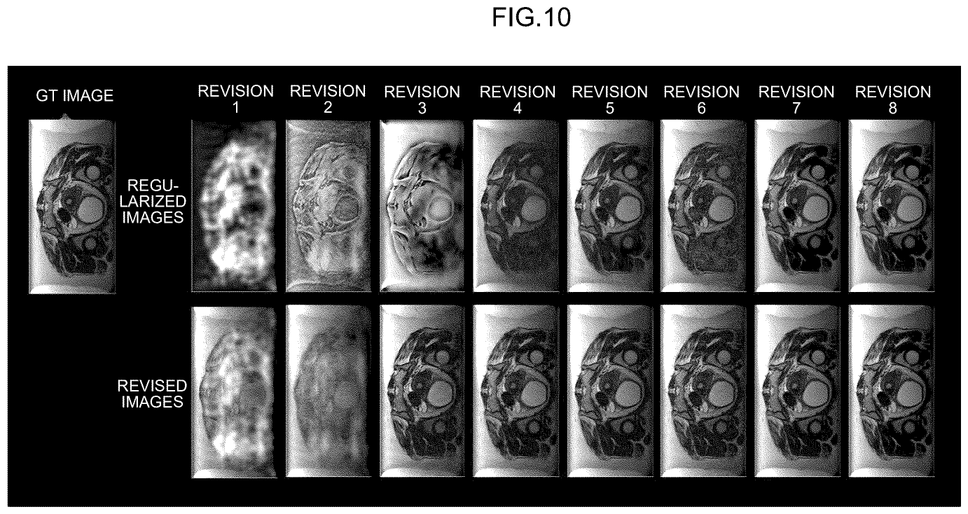

FIG. 10 is a drawing for explaining examples of the regularized image data and the revised image data generated in the revision processes in the magnetic resonance image reconstruction method according to the embodiment. FIG. 10 is a drawing illustrating an example in which the axial plane in the vicinity of the prostate illustrated in FIG. 9 is reconstructed by implementing the magnetic resonance image reconstruction method according to the embodiment.

As illustrated in FIG. 10, in the magnetic resonance image reconstruction method according to the embodiment, the revision process is performed eight times in total. Of those, the regularized image data generated in the revision processes performed for the first time and the second time exhibits artifacts and noise caused by the neural network. However, because the deviation correction is performed on the regularized image data exhibiting the artifacts and the noise, the subsequent revision processes are not impacted by the artifacts and the noise that occurred in the revision processes performed for the first time and the second time. Thus, the revised image data has converged in the revision process performed for the fifth time.

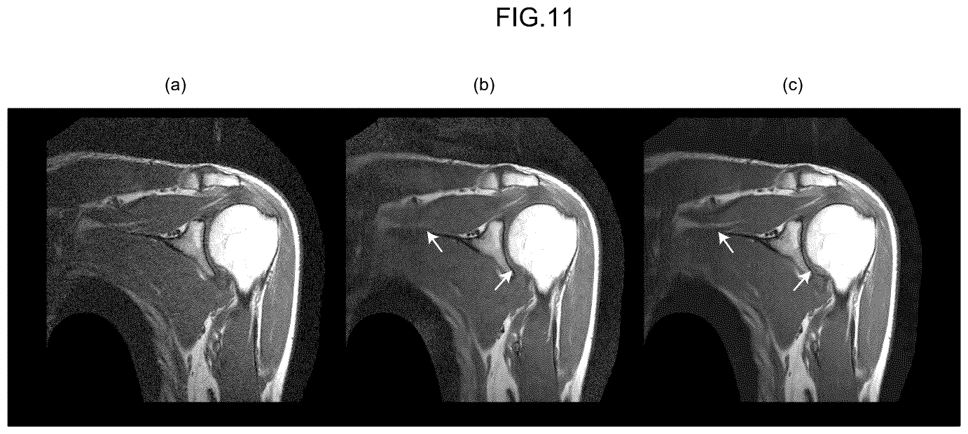

FIG. 11 is a drawing for comparing a magnetic resonance image reconstructed by implementing the magnetic resonance image reconstruction method according to the embodiment, with a magnetic resonance image reconstructed according to the comparison example.

(a) of FIG. 11 presents a local ground truth image of a knee part. (b) of FIG. 11 presents a local image of the knee part reconstructed by implementing the magnetic resonance image reconstruction method according to the comparison example. (c) of FIG. 11 presents a local image of the knee part reconstructed by implementing the magnetic resonance image reconstruction method according to the embodiment.

As apparent from FIG. 11, the local image of the knee part reconstructed by implementing the magnetic resonance image reconstruction method according to the comparison example exhibits remarkable artifacts in an upper part and a lower part of the image, while fine structures (in the positions indicated by the arrows) at the sites indicated by the arrows were not reconstructed. In contrast, in the local image of the knee part reconstructed by implementing the magnetic resonance image reconstruction method according to the embodiment, there is no remarkable artifacts or noise in the image, and the fine structures at the sites indicated by the arrows have been reconstructed.

When 4× undersampling is carried out, because the reconstruction implementing the magnetic resonance image reconstruction method according to the embodiment has relatively high stability, the magnetic resonance image reconstruction method according to the embodiment exhibits, in tests performed on various sites such as a head part, a chest part, the spine, and the lower back, a higher average Structural Similarity Index (SSIM) and a higher average Peak Signal-to-Noise Ratio (PSNR), as compared to those from the magnetic resonance image reconstruction method according to the comparison example.

A method for training the neural network and evaluating capabilities thereof

In the explanation above, the magnetic resonance image reconstruction method and the magnetic resonance image reconstructing apparatus according to the embodiments use the first neural network 61, the second neural network 71A, and the data integrity coefficient λ. These neural networks and the parameters need to be trained in advance to achieve normal operation. In the following sections, a method for training the neural networks and the parameters described above will be explained.

To begin with, a plurality of sets of training data stored in the storage unit 40 in advance are read. The sets of training data include the undersampled k-space data K₀ serving as input data, the mask data M corresponding thereto, and ground truth image data serving as output data.

Subsequently, the plurality of sets of training data are divided into training sets, test sets, and cross validation sets. For example, the ratio among the training sets, the test sets, and the cross validation sets may be 80%, 10%, and 10% or may be 908, 5%, and 58. For example, when the total number of sets of training data is 10000, the pieces of training data numbered as data #1 to #10000 may be divided into the training sets numbered as data #1 to #8000, the test sets numbered as data #8001 to #9000, and the cross validation sets numbered as data #9001 to #10000. In this situation, the input data within the sets of training data in the training sets is input to the magnetic resonance image reconstructing apparatus 1, so as to calculate estimate values for the ground truth image data by executing the magnetic resonance image reconstruction method according to the embodiment. After that, difference values are calculated between the estimated values for the ground truth image data and the ground truth image data, so as to carry out backpropagation on the basis of the difference values. In this manner, parameters for the neural networks and other machine-learnable parameters are changed so as to minimize the difference values between the estimated values for the ground truth image data output by the magnetic resonance image reconstructing apparatus 1 and the ground truth image data. The abovementioned procedure is repeatedly performed until the difference values between the estimated values for the ground truth image data output by the magnetic resonance image reconstructing apparatus 1 and the ground truth in the ground truth image data become smaller than a threshold value set in advance with respect to a great majority of the pieces of data in the test sets. At this point in time, it is determined that the training of the neural networks and the parameters have been completed.

After that, input data from the cross validation data (data #9001 to #10000) is input to the magnetic resonance image reconstructing apparatus 1 that has already been trained, so as to calculate, as evaluation data, a peak signal-to-noise ratio of the estimated values for the ground truth image data output by the magnetic resonance image reconstructing apparatus 1 and a structural similarity index between the estimated values and the ground truth image data.

In the above embodiments, the example was explained in which the image space regularizing unit, the deviation correcting unit, the data integrity processing unit, and the output unit of the present disclosure are realized as the image space regularizing unit 60, the deviation correcting unit 70 or 70A, the data integrity processing unit 80, and the output unit 90 explained in the above embodiments;

however, possible embodiments are not limited to this example. For instance, instead of being realized as the image space regularizing unit 60, the deviation correcting unit 70 or 70A, the data integrity processing unit 80, and the output unit 90 explained in the above embodiments, the image space regularizing unit, the deviation correcting unit, the data integrity processing unit, and the output unit of the present disclosure may be realized as a processing unit having the same functions with the use of hardware alone, software alone, or a combination of hardware and software.

Further, in the above embodiments, the processing units such as the image space regularizing unit 60, the deviation correcting units 70 and 70A, the data integrity processing unit 80, and the output unit 90 may be realized by using processing circuitry such as one or more processors, for example. In that situation, processing functions of the processing circuitry may be stored in the storage unit 40, in the form of computer-executable programs, for example. Further, the processing circuitry may be configured to realize the processing functions corresponding to the programs, by reading and executing the programs from the storage unit 40. In other words, when the corresponding processing circuitry has read the programs, the processing units have the constituent elements illustrated in FIGS. 1 and 5. Further, although the example was explained in which the programs corresponding to the processing functions of the processing circuitry are stored in the single storage unit, possible embodiments are not limited to this example. For instance, the programs corresponding to the processing functions may be stored in a plurality of storage units in a distributed manner, so that the processing circuitry is configured to read and execute the programs from the storage units.

Further, in the above explanation, the processing units such as the image space regularizing unit 60, the deviation correcting units 70 and 70A, the data integrity processing unit 80, and the output unit 90 are each realized by using a single piece of processing circuitry; however, possible embodiments are not limited to this example. For instance, the processing units may be structured by combining together a plurality of pieces of independent processing circuitry, so that the processing functions are realized as a result of the pieces of processing circuitry executing the programs. Further, the processing functions of the pieces of circuitry and the functional units may be realized as being distributed among or integrated into one or more pieces of processing circuitry, as appropriate. Furthermore, the processing functions of the pieces of circuitry and the functional units may be realized by a combination of hardware such as circuitry and software.

The term “processor” used in the above explanations denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)). When the processor is a CPU, for example, one or more processors are configured to realize the functions by reading and executing the programs saved in a storage unit. In contrast, when the processor is an ASIC, for example, instead of having the programs saved in the storage unit, the functions are directly incorporated as logic circuitry in the circuitry of one or more processors. The processors in the present embodiments do not each necessarily have to be structured as a single piece of circuitry. It is also acceptable to structure one processor by combining together a plurality of pieces of independent circuitry so as to realize the functions thereof. Further, it is also acceptable to integrate two or more of the constituent elements illustrated in FIG. 1 into one processor so as to realize the functions thereof.

In this situation, the programs executed by the one or more processors may be provided as being incorporated in advance in a Read-Only Memory, a storage unit, or the like. The programs may be provided as being recorded in a file in a format that is either installable or executable for those apparatuses, on a computer-readable storage medium such as a Compact Disk (CD)-ROM, a Flexible Disk (FD), a CD-Recordable (CD-R), or a Digital Versatile Disk (DVD). Further, the programs may be stored in a computer connected to a network such as the Internet, so as to be provided or distributed as being downloaded via the network. For example, the programs are structured with modules including the functional units described above. In the actual hardware, as a result of a CPU reading and executing the programs from a storage medium such as a ROM, the modules are loaded into a main storage apparatus so as to be generated in the main storage apparatus.

Further, the constituent elements of the apparatuses illustrated in the drawings in the above embodiments are based on functional concepts. Thus, it is not necessarily required to physically configure the constituent elements as indicated in the drawings. In other words, specific modes of distribution and integration of the apparatuses are not limited to those illustrated in the drawings. It is acceptable to functionally or physically distribute or integrate all or a part of the apparatuses in any arbitrary units, depending on various loads and the status of use. Further, all or an arbitrary part of the processing functions performed by the apparatuses may be realized by a CPU and a program analyzed and executed by the CPU or may be realized as hardware using wired logic.

Furthermore, with regard to the processes explained in the above embodiments, it is acceptable to manually perform all or a part of the processes described as being performed automatically. Conversely, by using a publicly-known method, it is also acceptable to automatically perform all or a part of the processes described as being performed manually. Further, unless noted otherwise, it is acceptable to arbitrarily modify any of the processing procedures, controlling procedures, specific names, and various information including various types of data and parameters that are presented in the above text and the drawings.

According to at least one aspect of the embodiments described above, it is possible to enhance stability of the image reconstruction.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A magnetic resonance image reconstruction method for reconstructing magnetic resonance image data on a basis of undersampled k-space data, the magnetic resonance image reconstruction method comprising:
generating second image data by performing a regularization process within an image space while using a first neural network on first image data generated on the basis of the undersampled k-space data;
generating third image data by correcting the second image data so that a pixel value statistical feature of the second image data approximates a pixel value statistical feature of the first image data;
generating fourth image data by performing a data integrity process on the third image data so that k-space data corresponding to the third image data approximates the undersampled k-space data; and
outputting image data based on the fourth image data as the magnetic resonance image data that has been reconstructed.

2. The magnetic resonance image reconstruction method according to claim 1, wherein generating the third image data includes: calculating a first parameter related to the pixel value statistical feature of the first image data and a second parameter related to the pixel value statistical feature of the second image data; and generating the third image data by correcting the second image data on a basis of the first parameter and the second parameter.

3. The magnetic resonance image reconstruction method according to claim 2, wherein generating the third image data includes: calculating deviation data indicating a deviation between the second image data and the first image data on the basis of the first parameter and the second parameter; and
generating the third image data by correcting the second image data on a basis of the deviation data.

4. The magnetic resonance image reconstruction method according to claim 3, wherein generating the third image data includes: calculating a value obtained by dividing the second parameter by the first parameter as the deviation data; and
generating the third image data by dividing the second image data by the deviation data.

5. The magnetic resonance image reconstruction method according to claim 2, wherein generating the third image data includes: generating the third image data by using a second neural network.

6. The magnetic resonance image reconstruction method according to claim 1, wherein the first neural network is a convolutional neural network.

7. A magnetic resonance image reconstructing apparatus that reconstructs magnetic resonance image data on a basis of undersampled k-space data and comprises processing circuitry configured:
to generate second image data by performing a regularization process within an image space while using a first neural network on first image data generated on the basis of the undersampled k-space data;
to generate third image data by correcting the second image data so that a pixel value statistical feature of the second image data approximates a pixel value statistical feature of the first image data;
to generate fourth image data by performing a data integrity process on the third image data so that k-space data corresponding to the third image data approximates the undersampled k-space data; and
to output image data based on the fourth image data as the magnetic resonance image data that has been reconstructed.
